(19) United States
(12) Patent Application Publication
(10) Pub. No.: US 2023/0203428 A1
(43) Pub. Date: Jun. 29, 2023

(54) METHODS OF IMPROVING INTRACELLULAR BIOMOLECULE EXTRACTION YIELD AND METHODS OF CELL LYSIS

(71) Applicants: Arul Mozhy VARMAN, Chandler, AZ (US); Dylan SMITH, Gilbert, AZ (US); Aditya SARNAIK, Tempe, AZ (US)

(72) Inventors: Arul Mozhy VARMAN, Chandler, AZ (US); Dylan SMITH, Gilbert, AZ (US); Aditya SARNAIK, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/147,630

(22) Filed: Dec. 28, 2022

Related U.S. Application Data
(60) Provisional application No. 63/294,314, filed on Dec. 28, 2021.

(51) Int. Cl. C12N 1/06; C12N 1/20

(52) U.S. Cl. CPC C12N 1/066; C12N 1/20

(57) ABSTRACT

The present invention relates to methods of improving cell lysis procedures and yields of intracellular biomolecules extracted from biomass. The methods comprise storing the microbial cells in ultra-low temperature (ULT) conditions for at least 10 minutes prior to lysing the cells.

### CROSS-REFERENCE TO RELATED APPLICATIONS

This application claims the benefit of U.S. provisional patent application 63/294,314, filed Dec. 28, 2021, the entirety of the disclosure of which is hereby incorporated by this reference.

### FIELD OF THE INVENTION

The invention relates to methods of microbial cell lysis and increasing the yield of an intracellular biomolecule extracted from microbial biomass.

### BACKGROUND OF THE INVENTION

Microbial genetic engineering has revolutionized the field of biotechnology for improving the production of economically viable inherent metabolite(s), heterologous biomolecules, and value-added chemicals. Ultra-low temperature (ULT) storage of the recombinant cells and harvested microbial biomass is a routine practice in biological laboratories before subsequent sample processing, but its impact on cell lysis efficiency and proteins of interest has been minimally reported. On the other hand, traditional storage techniques including freeze drying, and rapid chilling, which involve a freezing step are known to negatively impact bacterial survival over long duration. Additionally, freeze-thaw techniques are widely used for bacterial cell disruption. These recurring observations indicate the plausible derogatory impact of ULT on bacterial cell membrane and cell viability.

Cell lysis is an important step for the extraction of intracellular enzymes, peptides, and other biomolecules. Advances in protein engineering have rapidly accelerated our ability to engineer enzymes, enabled us to perform direct alterations in substrate specificity and enzyme activity. Hence, improvements in cell lysis procedure are crucial for the efficient extraction and purification of such enzymes. Presently, there are many commercial cocktails for cell lysis applications, including B-PER™ (Thermo Scientific™), CelLytic™ and BugBuster® (Sigma-Aldrich®), SoluLyse™ (Genlantis), as well as mechanical techniques, which exhibit efficient bacterial cell lysis capabilities. However, multiple parameters must be considered while devising an effective lysis strategy. Based on the microbial species and the composition of their cell envelopes, diverse lysis methods are employed. For example, mechanical cell disruption (a high-pressure homogenizer) is employed for thick-walled cells like microalgae; enzyme assisted cell lysis is used for plant cells; and detergents are used for animal cell lysis. In addition, Gram-positive bacteria with thick cell walls are notably difficult to lyse due to the presence of multiple layers of peptidoglycan polymers cross-linked by teichoic acid. On the contrary, Gram-negative cells have an outer membrane while cyanobacterial cells possess a thick exopolysaccharide layer. As a result of this anatomical variability, efficacious and less severe lysis strategies are required to obtain maximum yields of functional proteins. Employing harsh disruptive techniques can overcome the obstacles posed by complex bacterial envelopes, thereby successfully lysing the bacterial cells. Unfortunately, these lysis techniques can reduce the molecular functionality, which ultimately undermines the whole purpose of cell disruption.

Thus, there is a need for improvements in the cell lysis process that reduces the probability of experimental variations and preferably enhances extraction yield in subsequent sample processing.

### SUMMARY OF THE INVENTION

Described herein are methods of microbial cell lysis comprising storing a biomass comprising a plurality of microbial cells in ultra-low temperature (ULT) for 10 minutes to 7 days prior to lysing the plurality of microbial cells, wherein the storage in ULT prior to lysing the plurality of microbial cells increases the efficiency of the lysing step compared to lysing the plurality of microbial cells without storing the biomass in ULT prior to the lysing step. Thus, in some implementations, the method of microbial cell lysis comprises providing a biomass comprising a plurality of microbial cells; storing the biomass in ULT for 10 minutes to 7 days; and then lysing the plurality of microbial cells.

Also described herein are methods of increasing the yield of intracellular biomolecules extracted from a microbial biomass, for example, the yield of proteins, DNA, and/or RNA extracted from the microbial biomass. The method comprises providing a biomass comprising a plurality of microbial cells; storing the biomass in ULT for 10 minutes to 7 days before lysing the plurality of cells to produce a cell lysate; and then extracting an intracellular biomolecule from the cell lysate.

ULT ranges between −20° C. and −130° C. In certain implementations, the ULT is between −20° C. and −80° C. The disclosed methods can improve the lysis of and the protein extraction yield from microbial cells with a variety of cell envelope composition—whether it has a tough cell wall with a peptidoglycan layer or mixture of an extracellular matrix and thylakoid multi-membrane system. The yield in a protein extraction from either Gram-positive bacteria or Gram-negative bacteria would increase from storing the microbial cells in ULT. Storing these microbial cells in ULT would also improve lysis of these cells. In some aspects, the microbial cells are from Escherichia coli, Bacillus subtilis, or Synechocystis.

In certain implementations, the biomass is stored in ULT for about 10 minutes, about 120 minutes, a day, two days, three days, four days, five days, six days, or seven days. In particular implementations, the biomass is stored in −80° C. for about 120 minutes, a day, or two days.

### BRIEF DESCRIPTION OF THE DRAWINGS

FIG. 1 depicts, in accordance with certain embodiments, the effect of ultra-low temperature (ULT) storage on bacterial cell membrane integrity. The cells stored over a period of 7 days at ULT were subjected to a PI assay. Actively growing bacterial cell membranes pose a barrier for PI entry within the cell; however, it can penetrate through membrane compromised or dead bacterial cells and intercalate within their polynucleotides yielding active fluorescence. Bacterial cell envelopes were damaged with storage at ULT, thereby increasing PI entry within the cells and hence increased fluorescence. Highest damage was observed in Escherichia coli cells, followed by Bacillus subtilis and Synechocystis sp. PCC 6803. Cells stored with the medium displayed higher fluorescence than the cells stored without the culture medium. Data are shown as mean±S.D, n=3.

FIGS. 2A-2D depict, in accordance with certain embodiments, the impact of storing cells at ULT on cell viability. The cells stored at ULT over a period of 7 days were subjected to MTT assay. MTT assay is based on a simple principle of active dehydrogenases present in viable cells, whereby such active cells can convert MTT to MTT-formazan-cell complex (solubilized in DMSO), which has a λₘₐₓ of 550 nm. This establishes direct correlation between cell viability and absorbance at 550 nm. We hereby present MTT assay standard curves for bacteria with different cell densities (measured by Abs₆₀₀ or Abs₇₃₀) (FIG. 2A), and relative (%) viability plots (with respect to that of freshly harvested cells) for E. coli (FIG. 2B), B. subtilis (FIG. 2C), Synechocystis 6803 (FIG. 2D) with (blue) and without (orange) the culture media. Relative viabilities were also compared with their glycerol stocks (bars in the plots) which had almost 100% viability. Data are shown as mean±S.D, n=3.

FIGS. 3A-3C depict, in accordance with certain embodiments, the impact of ULT storage on protein extraction efficiency. FIG. 3A is a schematic representation of bacterial cell structures used in this study and the steps followed for protein extraction and analysis. Comparative protein concentrations of whole (FIG. 3B) and soluble fraction of (FIG. 3C) cell lysates from E. coli (yellow), Synechocystis 6803 (green) and B. subtilis (blue) over 7 days of ULT storage. E. coli displayed significantly highest protein yields. The experiments were performed in biological triplicates. Statistical significance was determined using t-test (p<0.05). Data represented as Mean±SD, n=3.

FIG. 4 depicts, in accordance with certain embodiments, the fluorescence analysis of the proteins extracted from E. coli. Fluorescence intensity of soluble protein lysates (bars) indicates increase in protein extraction efficiency with the duration of ULT storage. Quantification of functional proteins was performed using whole cell lysates to determine relative fluorescence intensity (dots) of protein samples. Relative fluorescence intensity was calculated as a ratio of proteins present in whole cell lysates from any day to that of day 0. The experiments were performed in biological triplicates. Statistical significance was determined using t-test (5% level of significance). Data represented as Mean±SD, n=3.

FIG. 5 depicts, in accordance with certain embodiments, the impact of short-term ULT storage on protein extraction efficiency and functionality. Comparative protein concentrations of the soluble fraction of cell lysates from E. coli strains expressing mCherry (red) and eGFP (green) over 0, 10, 30, 60, 120 mins of ULT storage. Both the E. coli strains displayed significant improvement in protein extraction after ULT storage. Fluorescence analyses further confirmed sustained protein functionality. The experiments were performed in biological triplicates. Statistical significance was determined using t-test (5% level of significance). Data represented as Mean±SD, n=3.

FIGS. 6A and 6B depict, in accordance with certain embodiments, a comparative analysis of −80° C. and −20° C. storage of E. coli biomass for a short-term period at 120 mins (FIG. 6A) and for long-term period at 24 h and 48 h (FIG. 6B) revealed that the lysis efficiency is relatively higher with −80° C. and −20° C. Both the storage temperatures yield higher protein concentration than the freshly harvested E. coli biomass with intact protein functionality indicated as relative (%) fluorescence intensities. Therefore, any ULT temperatures can be used while practically implementing the strategy for improving the bacterial cell lysis, based on laboratory norms and accessibility.

FIG. 7 depicts, in accordance with certain embodiments, compares the effect of −20° C. storage of E. coli biomass for short-term (120 mins) and long-term (24 h and 48 h) on protein extraction. E. coli (mCherry) strains exhibited 4.6-folds improvement in the concentration of extracted protein after 48 h, whereas E. coli (eGFP) showed 1.8-fold increase in the extracted protein concentration over that from the freshly harvested biomass. Relative (%) fluorescence intensity further supported our low temperature storage approach with sustained protein functionality.

### DETAILED DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “intracellular biomolecule” refers to molecules found within a cell, including any proteins produced by the cell and the cell's nucleic acids.

As used herein, the term “ultra-low temperature storage” or “ULT storage” refers to storing conditions of between −20° C. to −130° C. In some aspects, ULT storage includes storage in liquid nitrogen, which is between −120° C. and −130° C.

Cell lysis is an important unit operation for extraction of intracellular biomolecules including nucleic acid (DNA/RNA) and proteins. Currently, many commercial cell lysis reagents and mechanical techniques are available for efficient cell disruption, but multiple parameters should be considered while devising effective lysis strategy that leverages the predicted competency of these methods. Based on type of cells and the composition of their cell envelopes, specific lysis methods are employed; for example, mechanical cell disruption using high pressure homogenizer for thick-walled cells like microalgae, enzyme assisted cell lysis for plant cells, and animal cell lysis using detergents. Interestingly, bacterial cell walls are multilayered and hence are relatively difficult to break open. Correspondingly, need for cell lysis must be taken into account while selecting the disruption technique, as it becomes primary consideration while extraction of sensitive biomolecules, especially functional peptides and enzymes. Use of harsh techniques can successfully lyse the bacteria, but can equivalently reduce the molecular functionality, ultimately depreciating the whole purpose of cell disruption. On the other hand, fresh cultivation of bacterial cells for examining new parameters for the same in vitro assay; every time followed by lysing might lead to significant deviations in the results. Hence, it is necessary to narrow down to an experimental strategy for effective cell lysis along with retaining the protein functionality over a longer time duration.

It is important to investigate the impact of cell composition and the duration of ULT storage to identify their combined effect on cell lysis efficiency. As it is known that freezing cells lead to decrease in cell viability, that storing microbial biomass at ULT can provide the added benefit of improving cell lysis efficiency by avoiding harsh treatments. On the other hand, cultivating fresh cells several times to examine various enzyme parameters can lead to significant deviations in the results owing to experimental variability or manual errors.

Disclosed herein is an improved method of cell lysis based on improving cell disruption. The improved cell disruption procedures developed incorporate a ULT incubation step. The method was verified across different types of bacteria. The freezing step improves cell lysis regardless of the type of cell envelope, cell membrane, or cell wall composition. Lysis of both cells having a tough cell wall with a peptidoglycan layer or a mixture of an extracellular matrix and thylakoid multi-membrane system have been shown herein to benefit from the freezing step. The disclosed method is applicable to Gram-positive bacteria and Gram-negative bacteria, for example, Escherichia coli, Bacillus subtilis, or Synechocystis. Based on the cell wall composition, the type of lysis solution can be changed, but disclosed method can bring about significant improvement in the conventional lysing procedure. The method of cell lysis described herein improves the efficiency of cell lysis for any lysis method, including but are not limited to, osmotic shock, ultrasonication, milling with glass beads, chemical lysis, enzymatic lysis, and thermal lysis.

The ULT incubation step comprises storing the cells for at least 10 minutes in sub-freezing conditions, for example between −20° C. to −130° C. In some aspects, the cells can be stored in −20° C. to −80° C. for two weeks prior to processing. In some embodiments, the methods of microbial cell lysis comprise storing a biomass comprising a plurality of microbial cells in ULT for 10 minutes to 7 days prior to lysing the plurality of microbial cells. In some implementations, the biomass may be stored in ULT for longer than 7 days to reduce errors that would occur due to experimental variations upon repetition. As shown in the examples, storage in ULT prior to lysing the plurality of microbial cells increases the efficiency of the lysing step compared to lysing the plurality of microbial cells without storing the biomass in ULT prior to the lysing step. Thus, in particular implementations, the described method of cell lysis comprises providing a biomass comprising a plurality of microbial cells; storing the biomass in ULT for 10 minutes to 7 days; and lysing the plurality of microbial cells.

Increased lysis efficiency releases more intracellular biomolecules (for example, proteins, nucleic acids, etc.) into the cell lysate. Accordingly, also described herein are methods of increasing the yield of an intracellular biomolecule extracted from microbial biomass. The method comprises providing a biomass comprising a plurality of microbial cells; storing the biomass in ULT for 10 minutes to 7 days; lysing the plurality of cells to produce a cell lysate; and extracting the intracellular biomolecule from the cell lysate.

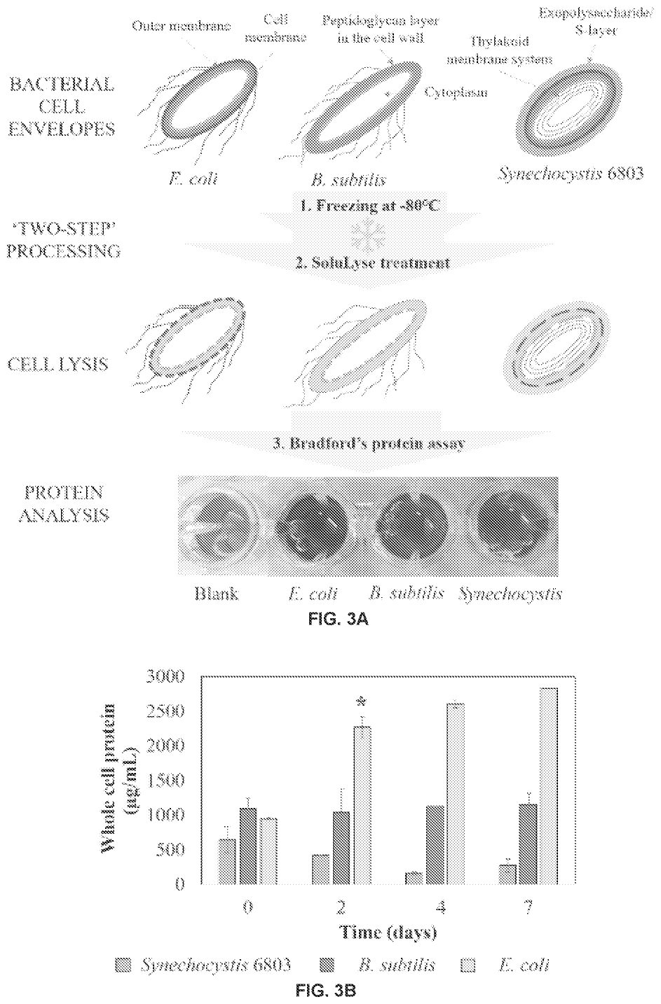

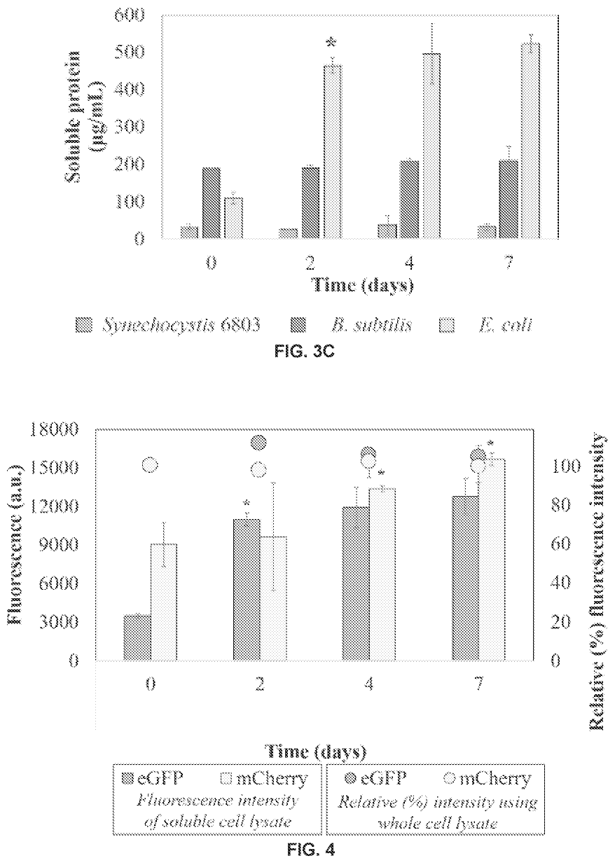

It was surprisingly discovered that storing microbial cells in ULT for a period of 10 minutes to as long as a week prior to lysing the microbial cells improved lysis efficiency and yield of protein extraction without negatively impacting the molecular functionality of the extracted protein (see FIGS. 4 and 5). In some aspects, the step of ULT storage doubles the amount of intracellular protein yielded from a conventional protein extraction process without affecting the function of the intracellular biomolecule. Thus, the methods described herein include methods of increasing the yield of extracted protein from microbial biomass. The method comprises providing a biomass comprising a plurality of microbial cells; storing the biomass in ULT for 10 minutes to 7 days; lysing the plurality of cells to produce a cell lysate; and extracting protein from the cell lysate. The described methods of increasing the yield of extracted protein from microbial biomass also improves functional protein extraction efficiency by storing microbial biomass at ULT for a short time and to use them for in vitro assays, his-tag purification, etc.

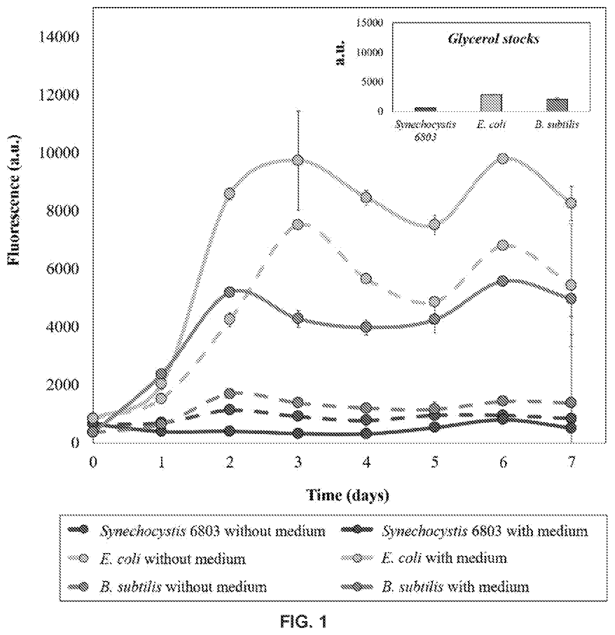

In certain implementations, the storage conditions are at −80° C. for 10 minutes, for 120 minutes, or for two days. In other implementations, the storage conditions are at −20° C. for 10 minutes, for 120 minutes, or for two days.

Where the biomass comprises tough cyanobacteria or Gram-positive cells, the lysis step of the disclosed methods of cell lysis and of increasing the yield of extracted protein further comprises supplementing the lysis solution with specific hydrolyzing enzymes, for example, cellulase, and lysozyme. As shown in FIG. 1, ULT storage with culture medium improved cell lysis of E. coli or B. subtilis when compared to ULT storage without medium but not for Synechocystis, which has the toughest cell well of the three microorganisms tested. Thus, lysis of microbial cells with complex cell envelope, such as Synechocystis, would further benefit from ULT storage with medium containing a lytic enzyme (for example, lysozyme, lysostaphin, zymolase, cellulose, protease, glycanase, chitinase, or pectinase).

### EXAMPLES

The present invention is further illustrated by the following examples that should not be construed as limiting. The contents of all references, patents, and published patent applications cited throughout this application, as well as the Figures, are incorporated herein by reference in their entirety for all purposes.

### I. Impact of ULT-Storage on Bacterial Cell Viability

ULT storage of harvested microbial biomass is a routine practice in biological laboratories. Biomass is usually stored during transport or long-term experimentation for subsequent processing of the samples. However, its impact on microbial cells is minimally reported. The effect of ULT storage on the chosen microbial platforms was estimated based on cell membrane integrity and viability using PI and MTT assays, respectively. The following three bacterial candidates with different cell envelope structure and composition were selected: E. coli (Gram-negative); B. subtilis (Gram-positive); and Synechocystis sp. PCC 6803 (Gram-negative cyanobacterium).

Cells were grown under their respective optimal culture conditions (see materials and methods), the biomass was harvested and frozen at −80° C. for defined time periods, and the corresponding results were correlated to the cell lysis efficiency. Cells were stored with and without culture medium to study the effect of frozen aqueous medium on cellular integrity.

The cells stored at ULT were subjected to a PI assay. PI is a fluorescent intercalating dye which is excluded by viable cells and is widely used for distinguishing live bacteria from the dead ones. Therefore, the PI assay could be used as an indicator of cell envelope integrity, especially the cell membrane. Our results indicated that within 2 days of storage, fluorescence was found to increase significantly for E. coli cells, followed by B. subtilis (FIG. 1). Synechocystis 6803 pellets did not display any significant change in the fluorescence over the storage duration.

Moreover, cells stored in the growth medium yielded higher fluorescence readings as compared to cells stored without the medium for both E. coli and B. subtilis (FIG. 1). This distinctive response could be attributed to the additional effect of extracellular media crystallization weakening the cell envelope. The assay confirmed that the bacterial cell membranes were compromised with increased storage under ULT. However, PI can pass through the cell envelopes of actively growing cells in miniscule amounts, but is actively exported, unlike cells with weakened cell walls or dead cells. It has also been demonstrated that PI might in some cases provide false dead signals correlating to high membrane potential due to cell physiology and not the membrane damage. Apart from this, unbound PI has been found to possess strong background fluorescence of 400-500. These background signals could not be prevented in fluorescence readouts but were instead negated by subtracting the signal obtained from the blank control. Considering these limitations of PI assay, the effect of ULT was further verified using MTT viability assay according to the protocol developed by Wang et al.

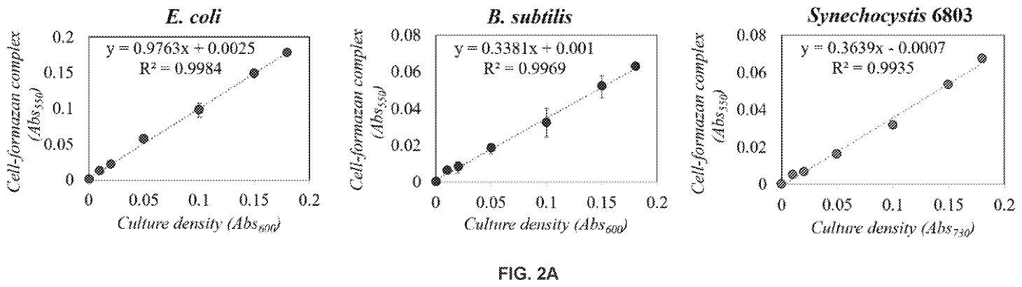

As a next step, the biomass of all three bacterial candidates stored at ULT were subjected to an MTT assay. In the MTT assay, viable cell density was primarily estimated from the standard curves (Abs₅₅₀ v/s Abs₆₀₀) of the corresponding species prepared from actively growing cells (FIG. 2A). Relatively high correlation coefficient (close to 1) indicated the reliability of using the standard curves for assessing cell viability.

Relative (%) cell viability based on MTT assay revealed that E. coli cells significantly lost their viability when incubated with the culture medium as compared to the cells stored as pellets (i.e., without the culture medium) within 24 hours of storage at −80° C. (FIG. 2B). This trend was not observed with the other two bacterial candidates and hence this can be attributed to the Gram-negative cell wall structure of E. coli and the rupture caused by extracellular shear forces originating from ice crystals. On the other hand, E. coli cell pellets retained significant viability for 3 days and the cell viability dropped over the next 4 days of storage. In correlation with the PI assay results, it can be stated that although the E. coli membrane integrity was found to be significantly compromised on day 2, the cell viability was still retained at ˜60% based on MTT assay results (FIGS. 1 and 2B). More importantly, both of these assays clearly indicated the negative impact of extracellular aqueous medium on the membrane integrity and cell viability (FIGS. 1 and 2B).

Like E. coli, B. subtilis cells exhibited effective viability for the first two days (60-80%), with and without the culture medium. Following the initial two days, % viability dropped to almost ˜40% on the 3rd day and to ˜10% by the 7th day (FIGS. 2B and 2C). This differential response to the presence of aqueous media by B. subtilis could be correlated to the presence of a relatively thick peptidoglycan layer in its cell wall (FIGS. 1 and 2C). The gradual loss of viability would be the result of cryodamage caused by intra- and extracellular crystallization during the storage and can also be attributed to osmotic pressure changes due to crystallization.

On the other hand, Synechocystis 6803 cells retained significant membrane integrity and cell viability over the entire duration of the experiment irrespective of storage with or without the medium (FIGS. 1 and 2D). Synechocystis 6803 has been demonstrated to retain ˜41.8% of photosynthetic quantum yield, even after freezing, which correlates with the current observation. Overall, these findings support the fact that storing cells at −80° C. negatively impacts cell viability to varying degrees depending on the structure and composition of the cell envelope. In past studies, it has been observed that Gram-negative organisms are more prone to cryodamage than Gram positive ones. However, the current study is not in complete agreement with this report: cyanobacteria, despite being Gram-negative, displayed highest survival and membrane rigidity owing to their distinct cellular architecture.

### II. Impact of ULT-Storage on Protein Extraction

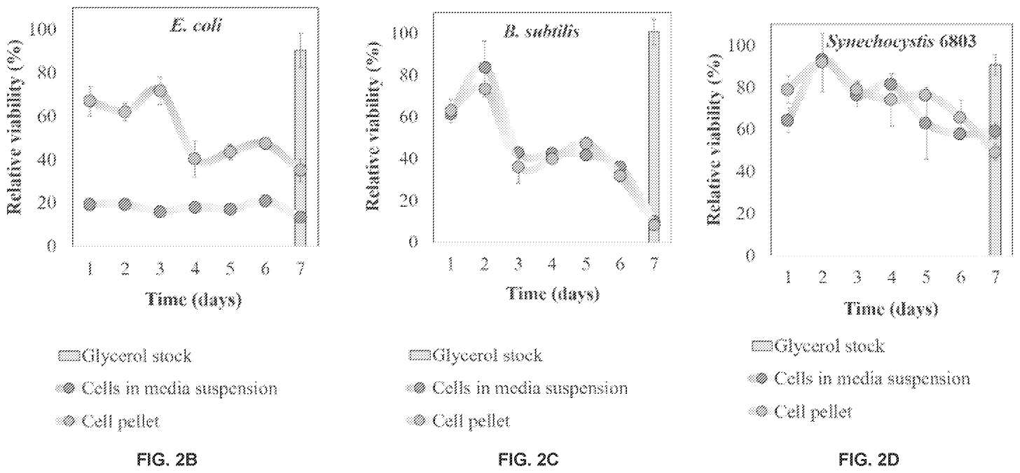

Reduced membrane integrity and decreased cell viability as observed in FIGS. 1 and 2a-2D during storage at −80° C. indicated that weakened cell envelopes may aid cell lysis and improve protein extraction efficiency. To verify this hypothesis, the impact of storing cells at −80° C. on protein extraction was evaluated. SoluLyse™ was used for protein extraction, and the protein concentrations in the resulting cell lysates were estimated using Bradford's assay (FIG. 3A).

Whole cell lysate possessing cell debris displayed higher protein concentration owing to the presence of insoluble protein fractions in comparison to the supernatant that contained only the soluble protein fraction (FIGS. 3B and 3C). In addition, an expected trend was observed between protein concentration (which is an indirect measure of cell lysis efficiency) and the cell envelope composition of bacterial cells. E. coli cells with relatively weaker cell envelope system displayed higher degree of cell lysis (i.e., higher protein concentration). Both B. subtilis and Synechocystis 6803 displayed only a miniscule cell breakage (FIG. 3C). As previously discussed, B. subtilis possesses a thick peptidoglycan layer and is relatively difficult to break-open, which is precisely reflected from the protein concentration in the cell lysate. Synechocystis 6803, on the other hand, has a unique extracellular matrix (S-layer) and multi-membrane (thylakoid) system within the cell that not only confers extended viability at −80° C., but is recalcitrant to the disruption steps employed to extract the intracellular proteins. Hence, it can be corroborated that along with external physical stress and the presence of an outer membrane, intracellular organelle architecture, like thylakoid membranes may influence cell morphology, cell survival, cell lysis and therefore, protein extraction efficiency. In addition, by comparing these results to FIGS. 1 and 2A-2D, it can also be concluded that there is a direct correlation between bacterial membrane integrity, cell viability during −80° C. storage and cell lysis efficiency, more significantly for E. coli cells.

### III. Impact of ULT-Storage on Protein Function

Storing cells in a frozen state negatively impacted cell viability enabling improved protein extraction. Although improving protein extraction is critical for several fields, it is equally important to make sure that the biomolecules are functional during the low temperature storage and post-extraction. As E. coli is the major microbial host used for protein expression studies, this part of the study was restricted only to E. coli strains. E. coli cells engineered to constitutively express fluorescent proteins mCherry and eGFP were used to estimate the effect of −80° C. storage on protein function. Total fluorescence from the whole cell lysates and from their soluble fractions (supernatant) were estimated. Relative fluorescence was calculated for the whole cell lysate, as a percentage of protein extracted from freshly harvested cells, to analyze the effect of ULT-storage on the protein function. Fluorescence estimation with the whole cell lysates indicated that the relative (%) fluorescence was maintained at an average of 100±3% for both the proteins (FIG. 4; dots) across all time points. This observation confirmed that the protein function was not affected during the entire storage period of the microbial biomass. In addition, fluorescence intensity (a.u.) of the soluble protein fraction (FIG. 4; bars) precisely correlated with the protein concentration trend observed in FIG. 3C showing proper extraction of intracellular soluble proteins. Therefore, storing cells at ULT can be harnessed for long term protein storage as the function of the protein within the unruptured cell is intact. Plus, it can provide the added benefit of obtaining improved protein yield from the cells stored at ULT.

### IV. ULT Storage is a Practical Step to Improve Protein Extraction

E. coli cells exhibited improved cell lysis and protein yield just within 2 days of ULT storage (FIG. 3C). As ULT storage is a simple step, this could very well be utilized to improve protein (or other biomolecules) extraction efficiency. However, storing cells for 2 days with the goal to improve protein extraction from cells is not time efficient. To understand if a shorter more practical storage time exists, the effect of short-term ULT storage on protein extraction efficiency was evaluated using E. coli transformants expressing fluorescent proteins for ULTS storage periods of less than 2 days.

Interestingly, storing E. coli strains expressing mCherry just for 10 and 120 minutes resulted in 2.7-fold (from 93 μg/ml to 254 μg/ml) and 4-fold (93 μg/ml to 380 μ/ml) improvement in the protein extraction efficiency, respectively (FIG. 5). Likewise, storing E. coli strains expressing eGFP for 120 minutes resulted in approximately 2-fold (310 μg/ml to 602 μg/ml) increase in protein extraction (FIG. 5). In addition, as expected from FIG. 4, fluorescence analysis indicated the protein function to be intact (FIG. 5).

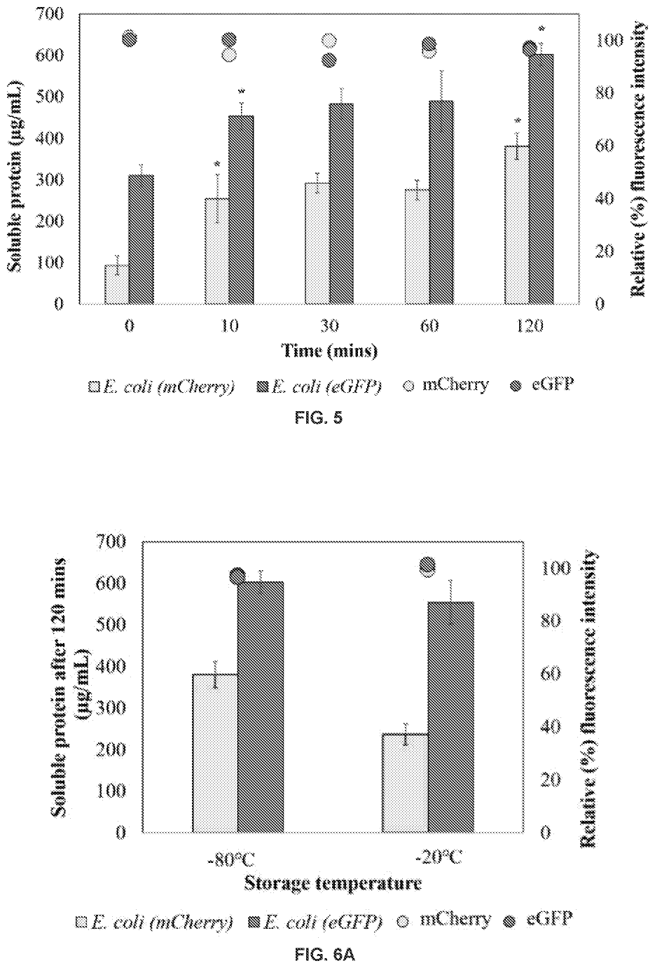

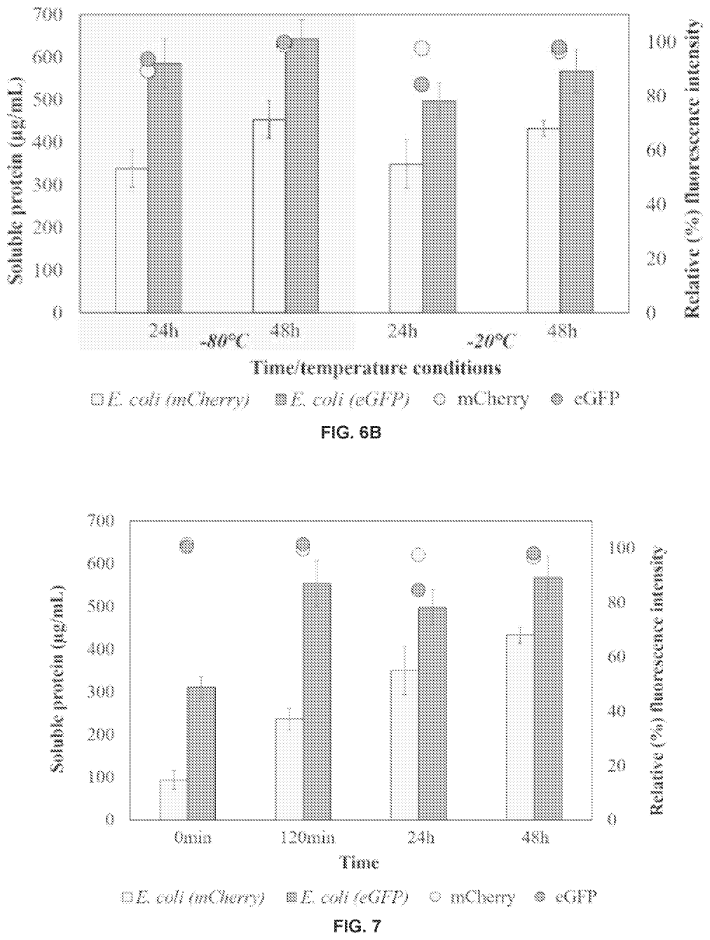

−20° C. storage is another common storage temperature employed in biological laboratories and it has been proven to have detrimental effect on bacterial cell viability. Therefore, a short comparative study was conducted between −80° C. and −20° C. storage of microbial cells. The results indicated that −20° C. storage yielded relatively less amount of protein from E. coli cells during both short-term (<120 mins) as well as long-term (>1 day) storage (FIGS. 6A and 6B). However, the extracted protein concentration is substantially higher than that from the freshly harvested cells, without significantly impacting the protein functionality (FIG. 7). Therefore, either of these low temperatures can be practically implemented for the storage of bacterial cell and to improve protein extraction. Overall, these findings suggest that a more practical ULT storage time of 10 to 120 minutes can be adapted by researchers to improve protein extraction efficiency by several folds.

### V. Methods and Materials

a. Chemicals and Reagents

Thiazolyl blue tetrazolium bromide (MTT), culture media components, and all the other chemicals were purchased from Sigma-Aldrich (St. Louis, Mo., USA). SoluLyse™ was purchased from Genlantis (San Diego, Calif.). Bradford's reagent was purchased from BioRad. Propidium iodide (PI) was purchased from G-Biosciences, Geno Technology Inc (St. Louis, Mo., USA).

b. Bacterial Strains and Cultivation

E. coli and B. subtilis were cultivated in LB medium at 37° C. and 250 rpm for 18 hours. Synechocystis sp. PCC 6803 (also referred to herein as “Synechocystis 6803”) was grown in BG-11 medium at 30° C. and 250 rpm under 100 μmol/m²/s light intensity for 4 days. These bacterial cells were subjected to PI assay and MTT viability assay. E. coli BL21-DE3 transformants expressing mCherry fluorescent protein (BBa_K2033011 plasmid possessing ampicillin resistance; 100 μg/mL) and E. coli DH5a transformants expressing eGFP (pZE27GFP-Addgene plasmid #75452 possessing kanamycin resistance; 25 μg/mL), constitutively, were cultivated as above and used for estimating cell lysis efficiency (along with B. subtilis and Synechocystis 6803) and the impact of ULT storage on protein function.

c. ULT-Storage

Bacterial cells (E. coli, B. subtilis, and Synechocystis 6803) were grown under the respective cultivation conditions. 5 mL of the cells were harvested, and their cell densities were adjusted to the optical density of 0.15 (for 200 μL) and centrifuged at 12,000 g for 5 min. One set of cell pellets was incubated with 200 μL of culture medium and another without the medium, followed by incubating in −80° C. freezer for up to 7 days. The PI assay, MTT assay, and fluorescence analyses were performed over 7 days in triplicates. Glycerol stocks (containing 20% sterile glycerol) were stored as the experimental positive control and analyzed along with the experimental samples at the end of their storage.

d. PI Assay

Bacterial cell densities (for E. coli, B. subtilis, and Synechocystis 6803) were adjusted to the Abs₆₀₀=0.15. Cells were stored with and without the culture media (200 μL) in −80° C. Freshly harvested cells and ULT stored samples obtained at different time points were analyzed for their membrane integrity using propidium iodide (PI) assay. 3 μM PI solution was made in nuclease-free water. Samples with the medium were thawed and centrifuged at 12000 g for 1 min. All the cell pellets were suspended in 200 μL of PI solution and dispensed into 96-well plates. The plates were incubated at 37° C. for 5 min by shaking at 3 mm amplitude. Spectrofluorometer Infinite1200 by TECAN was used for fluorescence analysis. The fluorescence was obtained at the excitation wavelength of 530 nm and emission wavelength of 610 nm against the 3 μM PI solution which served as a blank control. The data were plotted as fluorescence against the number of days.

e. MTT Assay

MTT assay was performed following the protocol developed by Wang et al. Cell pellets were mixed with 20 μL of 5 g/L (w/v) MTT solution in water and immediately incubated at 37° C. for exactly 20 min. Dehydrogenase catalyze the reduction of MTT to MTT-formazan-cell complex, which could be prominently observed as the purple particles in the suspension. The resulting solution was centrifuged at 12,000 g for 2 min and the pellet was suspended in 500 μL DMSO followed by vortexing for 5 min yielding a magenta-colored solution. 40 μL of this solution was diluted with 160 μL DMSO and the absorbance was measured at 550 nm. As the optical density of the formazan complex directly corresponds to the number of viable cells, standard curves were generated for all the selected bacteria by using different cell densities (Abs₆₀₀=0.01, 0.02, 0.05, 0.10, 0.15, 0.18) that were prepared from cells in their exponential growth phase. The viable cell densities from the MTT assay were deduced by using the calibration curve corresponding to the bacterium (FIG. 2A).

f. Protein Extraction and Quantitation

To investigate the effect of long-term ULT storage, bacterial pellets were stored in −80° C. for 7 days, same as previously mentioned. ULT frozen cells were thawed at room temperature (RT) for 10 min. Fresh and/or ULT frozen cell pellets were mixed with 50 μL SoluLyse™ and slowly vortexed for 10 min. 150 μL of distilled water was added to these lysates. One set of lysates was centrifuged at 15,000 g for 2 min to obtain soluble protein fraction as the supernatant. Protein concentrations were estimated for both, whole (uncentrifuged) cell lysates and centrifuged cell lysates using Bradford's assay and the lysates of E. coli transformants were further used for fluorescence studies. To investigate the effect of short-term ULT storage, E. coli cells expressing fluorescent protein were stored in −80° C. for different time durations (10, 30, 60, 120 mins). The proteins were extracted as described before and quantified via Bradford's assay.

Furthermore, comparative effect of short-term (120 mins) and long-term (24 h, 48 h) storage at −20° C. on cell lysis and protein functionality was also performed and has been reported in the (FIGS. 6A, 6B, and 7).

g. Fluorescence Spectroscopy

E. coli transformants expressing the fluorescent proteins mCherry and eGFP were used as a surrogate to study the impact of ULT storage on protein function. mCherry fluorescence was estimated with excitation at 587 nm and emission at 630 nm, whereas eGFP fluorescence was estimated with excitation at 488 nm and emission at 507 nm using spectrofluorometer Infinite1200 by TECAN. Fluorescence analysis was performed with 200 μL of freshly harvested intact cells as well as cell lysates (whole and centrifuged) in 96-well plates.

h. Statistical Analysis

Mean values and standard deviations were calculated by Microsoft Excel standard functions. P-values used for determining statistical significance of our results were calculated in Microsoft Excel using Student's t-test.

What is claimed is:

1. A method of increasing the yield of an intracellular biomolecule extracted from microbial biomass, the method comprising:
providing a biomass comprising a plurality of microbial cells;
storing the biomass in ultra-low temperature (ULT) for at least 10 minutes;
lysing the plurality of microbial cells to produce a cell lysate after storage in ULT conditions; and
extracting the intracellular biomolecule from the cell lysate.

2. The method of claim 1, wherein the ULT is −80° C. to −20° C.

3. The method of claim 1, wherein the biomass is stored in ULT conditions for at least 120 minutes.

4. The method of claim 1, wherein the biomass is stored in ULT conditions for at least two days.

5. The method of claim 1, wherein the ULT conditions is −80° C. and the biomass is stored in ULT conditions for about 10 minutes.

6. The method of claim 1, wherein the ULT conditions is −80° C. and the biomass is stored in ULT conditions for at least 120 minutes.

7. The method of claim 1, wherein the ULT conditions is −80° C. and the biomass is stored in ULT conditions for about 2 days.

8. The method of claim 1, wherein the intracellular biomolecule is a protein.

9. The method of claim 1, wherein the microbial cells are from Gram-negative bacteria.

10. The method of claim 1, wherein the microbial cells are from Gram-positive bacteria.

11. The method of claim 1, wherein the biomass comprises a culture selected from the group consisting of: Escherichia coli, Bacillus subtilis, and Synechocystis sp. PCC 6803.

12. The method of claim 1, wherein the plurality of cells is lysed with a solution comprising a hydrolyzing enzyme.

13. A method of microbial cell lysis, the method comprising:
providing a biomass comprising a plurality of microbial cells;
storing the biomass in ultra-low temperature (ULT) conditions for at least 10 minutes; and
lysing the plurality of microbial cells,
wherein storing the biomass in ULT conditions prior to lysing the plurality of microbial cells increases the efficiency of the lysing step compared to lysing the plurality of microbial cells without storing the biomass in ULT conditions prior to the lysing step.

14. The method of claim 13, wherein the ULT conditions is −80° C. to −20° C.

15. The method of claim 13, wherein the biomass is stored in ULT conditions for about 10 minutes, about 120 minutes, one day, two days, three days, four days, five days, six days, or seven days.

16. The method of claim 13, wherein the ULT conditions is −80° C. and the biomass is stored in ULT conditions for at least 10 minutes.

17. The method of claim 13, wherein the ULT conditions is −80° C. and the biomass is stored in ULT conditions for at least two days.

18. The method of claim 13, wherein the microbial cells are from Gram-negative bacteria.

19. The method of claim 13, wherein the microbial cells are from Gram-positive bacteria.

20. The method of claim 13, wherein the biomass comprises a culture selected from the group consisting of: Escherichia coli, Bacillus subtilis, and Synechocystis sp. PCC 6803.
